(19) United States
(12) Patent Application Publication
(10) Pub. No.: US 2005/0214388 A1
(43) Pub. Date: Sep. 29, 2005

(54) Multivitamin formulations containing controlled-release magnesium

(76) Inventors: Thomas R. Gorham, St. Michael, MN (US); Lori M. Freese, Monticello, MN (US)

Correspondence Address: Daniel C. Schulte;Kagan Binder, PLLC, Suite 200, 221 Main Street North, Stillwater, MN, 55082, US

(21) Appl. No.: 11/058,841

(22) Filed: Feb. 15, 2005

Related U.S. Application Data
(60) Provisional application No. 60/545,405, filed on Feb. 18, 2004.

(51) Int. Cl. A61K 33/08; A61K 31/525

(52) U.S. Cl. 424/692; 514/251; 424/468; 514/52; 514/350

(57) ABSTRACT

Described are dosage forms that contain magnesium in a therapeutic, controlled release form, wherein the dosage form includes one or more additional vitamin or mineral ingredients such as folic acid compound, vitamin B₆, vitamin B₁₂, calcium, magnesium, boron, as well as methods of administering such formulations.

### PRIORITY CLAIM

The present non-provisional Patent Application claims the benefit of priority under 35 USC 119 from commonly owned United States Provisional Patent Application having Ser. No. 60/545,405, filed on Feb. 18, 2004, in the name of Gorham et al., and titled MULTIVITAMIN FORMULATIONS CONTAINING CONTROLLED-RELEASE MAGNESIUM, which Patent Application is incorporated herein by reference in its entirety.

### FIELD OF THE INVENTION

The invention relates to oral dosage forms that include therapeutic magnesium in a controlled release form, and related methods, wherein a dosage form also contains another vitamin or mineral in non-controlled release form, such as a folic acid compound, vitamin B₆, or vitamin B₁₂.

### BACKGROUND

Multivitamin formulations are popular for supplementing dietary vitamins and minerals. Many varieties of multivitamin formulations are available, and some contain different combinations and amounts of two or more vitamins and minerals such as folic acid, vitamin B₁₂, vitamin B₆, vitamins A, C, D, E, K, and minerals such as boron, calcium, magnesium, iron, etc.

Magnesium is a mineral considered to be essential in the human body for normal metabolic functions. Magnesium has been identified as important in certain specific body functions or conditions such as bone health, cardiovascular health (e.g., relating to hypertension), and in treating diabetes.

With respect to healthy bones, there is growing evidence that magnesium may play an important role in qualitative bone changes, and magnesium deficiency may be a risk factor for postmenopausal osteoporosis. Adequate magnesium levels are also believed to be important for proper calcium metabolism.

With respect to cardiovascular health, magnesium depletion may raise blood pressure and may lead to cardiovascular complications such as cardiac ischemia, ECG changes, and arrhythmia. Some research has demonstrated a mild blood pressure lowering effect of magnesium supplementation, although results have been conflicting.

With respect to diabetes, some studies have demonstrated that magnesium depletion may result in insulin resistance and impaired insulin secretion, thereby worsening glycemic control in diabetic patients. Dietary magnesium supplements have been shown to improve glucose tolerance and improve insulin response in the elderly.

Thus, treatment or prevention of magnesium deficiency can effect multiple healthful results. Unfortunately, magnesium intake can cause undesirable gastrointestinal side-effects such as diarrhea or nausea.

In addition to magnesium, other vitamins and minerals are useful for administering to individuals to treat or prevent various specific medical conditions. Multivitamin formulations designed for such specific purposes may include selected amounts of specific vitamins and minerals believed to be of particular benefit to individuals having a specific medical condition. As a single example, multivitamin tablets containing folic acid, vitamin B₁₂, and vitamin B₆ are sold to reduce serum homocysteine and promote cardiovascular health. Other examples of popular multivitamin formulations sometimes contain vitamins and minerals including calcium, and may be taken for treating or preventing osteoporosis.

### SUMMARY

The invention relates to oral dosage forms that include therapeutic magnesium in a controlled release form, along with another vitamin or mineral in a non-controlled release form.

Dosage forms of the inventive subject matter overcome some of the deficiencies of currently-available dosage forms that contain magnesium, e.g., in the form of multivitamin nutritional supplements, by providing dosage forms that are designed to provide a therapeutic amount and form of magnesium in a controlled-release form. Providing magnesium in a controlled release dosage form can reduce or minimize the occurrence of or the severity of gastrointestinal side-effects caused by magnesium, and may preferably prevent or avoid gastrointestinal side-effects altogether. The dosage form may at the same time provide for one or more additional vitamins or minerals in a non-controlled release form. The additional vitamins or minerals may be included to treat or prevent general deficiencies in one or more vitamins or minerals. Alternately, one or more additional non-controlled release vitamin or mineral may be included in the inventive dosage forms to treat or prevent a specific condition.

According to the invention, magnesium is administered in a therapeutic, controlled release form. The amount and form of magnesium can be any that are therapeutically effective. The use of a controlled release magnesium dosage allows for administering magnesium while reducing the possibility or severity of common gastrointestinal side-effects such as nausea or diarrhea, and can also allow for relatively higher amounts of magnesium to be used while still reducing or minimizing the possibility or severity of side-effects. In certain preferred embodiments, a dosage form can be in the form of a capsule or a tablet, and controlled release magnesium can be in the form of magnesium oxide, which has a relatively high elemental magnesium content. This can allow for reduced dosage (e.g., tablet) size and can preferably allow for a dosage form that can be administered only once daily, but that still includes a desired relatively high dose of magnesium. Single daily administering of a dosage form is more convenient and easier for a patient to remember to administer, all of which improve patient compliance.

In exemplary embodiments, magnesium may be included in the dosage form in a total amount of from about 100 to about 600 milligrams (mg) of elemental magnesium per unit dose (dosage form), preferably greater than 250 milligrams elemental magnesium per unit dose, e.g., from 300 to 600 mg elemental magnesium per unit dose. Certain preferred dosage forms that include magnesium oxide as a therapeutic magnesium compound can include from 200 to 800 milligrams of controlled release magnesium oxide, e.g., from 300 to 750 milligrams controlled release magnesium oxide. In certain embodiments of the invention, the amount of magnesium can be sufficient to provide a daily requirement of magnesium in a single dosage form, for once a day administering.

The dosage forms may be used to treat or prevent magnesium deficiencies generally, or may be administered to treat or prevent specific conditions such as diabetes, degenerative bone disease such as osteoporosis, and cardiovascular conditions. The dosage forms and methods are preferably useful for treating mammals, especially humans.

A dosage form of the invention also preferably includes other vitamin or mineral ingredients, besides the controlled release therapeutic magnesium, but in a non-controlled release form. Examples of such vitamins and minerals include folic acid, vitamin B₆, and vitamin B₁₂. As a specific example, folic acid can be administered with magnesium to treat cardiovascular disease, diabetes, or degenerative bone disease. Even more specifically, an embodiment of the invention relates to dosage forms that contain controlled release magnesium in combination with non-controlled release folic acid compound, vitamin B₆, and vitamin B₁₂, e.g., to treat cardiovascular disease, diabetes, or degenerative bone disease such as osteoporosis.

Certain embodiments of the invention include active ingredients that include only controlled release magnesium (e.g., magnesium oxide), and non-controlled release folic acid compound, vitamin B₆, and vitamin B₁₂. For example, a dosage form may contain active ingredients that consist essentially of controlled release therapeutic magnesium and non-controlled release folic acid compound, vitamin B₆, and vitamin B₁₂, meaning that no more than insubstantial amounts of any other active vitamin, mineral, or pharmaceutical ingredient is included in the dosage form to function as an active vitamin, mineral, or pharmaceutical compound. Alternately, a dosage form may contain active ingredients that consist of therapeutic magnesium (e.g., magnesium oxide) and folic acid compound, vitamin B₆, and vitamin B₁₂, meaning that no other active vitamin, mineral, or pharmaceutically-active ingredient is included in a dosage form to act as active vitamin, mineral, or pharmaceutical compound.

The controlled release magnesium can be provided in any therapeutic form, and a controlled release profile can be produced by any useful dosage form construction. A controlled release portion of a dosage form may include, as examples, one or more of: a powder, a tablet, or a multiplicity of particles or beads, any of which contain therapeutic magnesium along with a controlled release agent; or pellets, beads, granules, or particles that are coated with a water insoluble coating and optionally contained within a capsule or a compressed tablet.

A non-controlled release portion of a dosage form can be prepared to release active ingredients (e.g., vitamins and minerals) according to a non-controlled release profile, by methods and dosage form constructions that will be understood. Similarly, the combination of a controlled release portion and a non-controlled release portion in a single dosage form can be accomplished by methods that will be understood.

One specific example of a solid dosage form of the invention is a multi-layer tablet having at least one layer containing therapeutic magnesium formulated for controlled release. The controlled release layer may include a compressed layer containing binder, therapeutic magnesium, and a controlled release agent. Another layer of the multi-layer tablet can contain ingredients (vitamins or minerals) formulated for non-controlled (e.g., immediate) release. As one specific example of such a dosage form, the invention contemplates a bi-layer tablet that includes a controlled release layer that contains a therapeutic magnesium compound, preferably magnesium oxide, and a non-controlled release layer that includes folic acid, and preferably also one or more of vitamin B₆ or vitamin B₁₂.

In one specific aspect the invention relates to a method of administering magnesium. The method includes orally administering a dosage form that contains non-controlled release folic acid compound, vitamin B₆, and vitamin B₁₂, and controlled release magnesium.

In another specific aspect the invention relates to an oral dosage form that includes non-controlled release folic acid compound, vitamin B₆, and vitamin B₁₂, and controlled release magnesium.

### DETAILED DESCRIPTION

The invention relates to oral dosage forms that include therapeutic magnesium in a controlled release form, and other vitamins or minerals in non-controlled release form.

“Therapeutic” magnesium, as discussed herein, is a form of magnesium administered to produce a therapeutic effect on the recipient (mammal, e.g., human) upon oral administration, and therefore is administered in an amount and form useful to treat or prevent magnesium deficiency or to treat or prevent a specific condition related to or caused by a magnesium deficiency. Accordingly, a therapeutic magnesium compound is a magnesium compound—generally a salt, oxide, or hydroxide compound—of elemental magnesium (Mg²⁺) with an oxygen (O⁻²), hydroxide ((OH⁻)₂), or other counterion (e.g., carbonate, phosphate, diphosphate, etc.), which at least partially dissociates upon exposure to the gastrointestinal tract to allow elemental magnesium to function metabolically. This means that the “therapeutic” magnesium is included in a dosage form in an amount and form to supplement dietary magnesium, and not as a component of a dosage form used to facilitate processing or administration of another active ingredient. As an example, therapeutic magnesium according to the invention does not include magnesium stearate when used in an amount effective to function as a lubricant in a controlled release dosage form for administering other (non-magnesium stearate) active ingredients (as is known). Nor does therapeutic magnesium include magnesium in the form of a counter-ion or ion associated with (e.g., chelated with) a different active pharmaceutical or otherwise metabolically-useful moiety.

Useful therapeutic magnesium compounds are known and include conventional pharmaceutically acceptable organic and inorganic dietary supplement salts of magnesium such as magnesium oxide, magnesium phosphate, magnesium diphosphate, magnesium carbonate, magnesium hydroxide, magnesium sulfate, magnesium gluconate, magnesium lactate, magnesium citrate, magnesium sulfate, magnesium aspartate, magnesium aspartate hydrochloride, magnesium chloride and the hydrates thereof, and the like. High magnesium content salts can be preferred, e.g., the hydroxide and oxide, because a final dosage form can therefore be less bulky. Magnesium oxide can be most preferred, because it has a relatively high percentage of elemental magnesium, and therefore can be preferred to minimize tablet size.

| MAGNESIUM | | |
|---|---|---|
| SALT | % MAGNESIUM | COMMON PRODUCTS |
| Magnesium | 5.9% | Magtrate 500 mg (29 mg elemental |
| Gluconate | | magnesium) |
| Magnesium | 60.3% | Uro-Mag 140 mg (84.5 mg elemental |
| Oxide | | magnesium) |
| | | MagOx 400 mg (241 mg elemental |
| | | magnesium) |
| Magnesium | 25.5% | Slo-Mag 535 mg (64 mg elemental |
| Chloride | | magnesium) |
| | | (Slo-Mag uses chloride hexahydrate |
| | | salt) |
| Magnesium | 12% | Mag-Tab SR (84 mg elemental |
| Lactate | | magnesium) |
| Magnesium | 16.2% | Unknown |
| Citrate | | |

Therapeutic magnesium can be included in a dosage form and administered in a controlled release manner, in any therapeutically useful amount, for general purposes of treating or preventing magnesium deficiencies or for treating or preventing any one of several specific conditions believed to be related to magnesium deficiency. Dietary magnesium is believed to play a role in the treatment or prevention of degenerative bone disease such as osteoporosis. Although decreased bone mass is a hallmark of osteoporosis, qualitative changes in bone are also present. There is growing evidence that magnesium may play an important role in qualitative bone changes. In addition, adequate magnesium levels are necessary for proper calcium metabolism.

Separately, magnesium has been mentioned as a possible factor relating to hypertension and blood pressure reduction and has also been mentioned in the treatment of diabetes.

Dietary intake studies of individuals have shown that magnesium intake can sometimes or often be well below the Recommended Daily Intake (RDI) established by the FDA for use in labeling (400 milligrams). This RDI corresponds to an upper limit in the form of supplements of 350 milligrams. The recommended upper limit for magnesium from supplements is established to minimize the incidence of diarrhea, which is the most common side-effect of magnesium supplementation. However, controlled release of therapeutic magnesium, as described herein, can allow for relatively higher amounts of magnesium (e.g., at least 350 milligrams of elemental magnesium, e.g., 580 milligrams of magnesium oxide) to be administered while still reducing or minimizing the possibility of gastrointestinal side-effects such as diarrhea. Also as mentioned, it can be preferable to administer a dosage form only once per day. According to the use of controlled release magnesium as described herein, with the preferred use of magnesium oxide as a therapeutic magnesium compound, a full day's amount of magnesium may be included in a dosage form for release over a given time after administering, without causing diarrhea.

As exemplary amounts of therapeutic magnesium included in a dosage form for controlled release according to the invention, an amount of elemental magnesium can be greater than 100 milligrams e.g., from 100 milligrams up to 2 grams or 5 grams per day, such as in the range from 100 to 600 milligrams elemental magnesium per dosage form. The amount of a therapeutic magnesium compound that corresponds to these ranges will depend on the mass of the non-magnesium portion of the therapeutic magnesium compound. As mentioned above, therapeutic magnesium compounds that include higher relative amounts of elemental magnesium (e.g., magnesium hydroxide, and especially magnesium oxide) can be preferred to reduce tablet size. In terms of magnesium oxide, preferred amounts can be in the range from 200 to 800 milligrams controlled release magnesium oxide per dosage form.

The therapeutic magnesium of the dosage form is in a controlled release form. As used herein, “controlled” release refers to a dosage form that releases therapeutic magnesium gradually over a controlled period of time after ingestion, preferably beginning in the stomach. Controlled release may also sometimes be referred to as “sustained” or “extended” release. A preferred controlled release profile according to the invention can be a release profile that reduces or minimizes, preferably prevents, the occurrence of gastrointestinal side-effects such as diarrhea, preferably while still allowing a desired amount of magnesium to be administered, e.g., from 100 to 600 milligrams elemental magnesium per dosage form.

As an exemplary controlled release profile, a total amount of controlled release therapeutic magnesium in a dosage form may be gradually released over a period of time that is at least 6 hours and that is less than 24 hours, e.g., gradually released over a period of time that is at least 8 hours and that is less than 12 hours. According to specific dosage forms and methods, this can mean that of a total amount of a controlled release therapeutic magnesium in a dosage form, an amount that is at least 70 percent (preferably 80 percent) of the total is released gradually over a time that is not less than 6 hours and that is not more than 24 hours. As a more specific example, an amount that is at least 70 percent (preferably 80 percent) of a total amount of controlled release therapeutic magnesium can be released over a time that is at least 8 hours and less than 12 hours. According to such preferred release profiles, magnesium is released gradually over a period of hours (e.g., up to 24 hours), starting immediately or nearly immediately (e.g., in the stomach). According to some specific controlled release profiles, 10 to 20 percent of a total amount of magnesium may become released within 3 hours from administering, 40 to 50 percent of total magnesium may be released within 12 hours from administering, and at least 80 or 90 percent is released within 24 hours.

Consistent with the present description, any mode of controlling the release of therapeutic magnesium from a dosage form, following administering, can be useful, including the use of known oral dosage forms that include compressed tablets or beads, or particles or granules coated with a water-insoluble material. Accordingly, preferred dosage forms may be in the form of compressed tablets or capsules that contain compressed or coated particles, either of which may be prepared by conventional procedures and using conventional ingredients including binders, controlled release agents, etc.

A dosage form of the invention most preferably can include at least one additional vitamin or mineral (in addition to the controlled release therapeutic magnesium), in a non-controlled release form. “Non-controlled release” (e.g., “immediate release”) refers to a dosage form that delivers the substantial entirety of a vitamin or mineral content in a non-controlled manner. The vitamin or mineral releases or is separated from the dosage form in a direct or relatively direct manner upon ingestion, e.g., in the stomach, without being substantially postponed by a component of the dosage form. Accordingly, the terms “non-controlled release” and “immediate release” refer to dosage forms that deliver the substantial entirety of a vitamin or mineral content in a non-controlled manner upon administration, as is consistent with the overall description herein.

As an example of a non-controlled release, a vitamin or mineral may begin release or separation from the dosage form within a matter of minutes, e.g., within 1 or 10 minutes. This typically means that a non-controlled release vitamin or mineral will experience initial release in the stomach. The non-controlled release vitamin or mineral can be substantially or completely released from a dosage form also within a matter of minutes, e.g., within 90 minutes, preferably within 45 minutes, more preferably within 30 minutes following ingestion. A preferred non-controlled-release vitamin or mineral ingredient can experience release or separation from the dosage form of at least 70 percent of the total amount of vitamin or mineral contained in the dosage form, preferably at least 75 percent, more preferably at least 80 percent, within 90 minutes, preferably 60 minutes and more preferably 45 minutes following ingestion.

One particular ingredient that can be a preferred non-controlled component of a dosage form according to the invention is a folic acid compound. The term “folic acid compound” is used in the present description in a manner consistent with its understood meaning in the vitamin and medical arts. The term refers to compounds generally understood to provide vitamin (folic acid) efficacy in its administered form or as a derivative of its administered form. The term refers to compounds commonly referred to as folic acid, folinic acid, folacin, tetrahydrate folate, and pteroyl monoglutamic acid, and more generally includes all pteroglutamates having vitamin activity.

The daily oral dosage of folic acid compound administered can, according to the invention, meet or exceed recommended daily intakes (the amount established by the FDA for use in labeling, “RDIs”) for certain individuals and perhaps for certain individuals having specific conditions. The RDI for folic acid is 400 mcg, generally, and 600 mcg for pregnant women. The table below lists the RDI of folic acid compound as well as selected other vitamins and minerals. According to the inventive methods and formulations, daily oral dosages can meet or exceed one or more of these RDIs. The inventive formulations can include, for example, folic acid compound in an amount greater than 400 micrograms (mcg), 500 mcg, 600 mcg, 1000 mcg, or 1500 mcg. Total daily dosage of folic acid compound, according to the invention, can be amounts greater than a RDI, and as described herein, can preferably be taken once in a single dosage form.

Recommended Intakes for Certain Vitamins and Minerals

| NUTRIENT | CURRENT RDI* | | | UPPER LIMIT (UL)** |
|---|---|---|---|---|
| Calcium | 1,000 mg (adults) | | | 2,500 mg |
| | (National Osteoporosis Foundation | | | |
| | recommendation 1,200 mg daily). | | | |
| Vitamin D | 400 IU (10 mcg) | | | 50 mcg (2,000 IU) |
| | NIH Recommends (Daily) | Men | Women | |
| | 19-50 years old | 5 mcg | 5 mcg | |
| | 51-70 years old | 10 mcg | 10 mcg | |
| | Over 71 years old | 15 mcg | 15 mcg | |

| | | |
|---|---|---|
| Folic acid | 400 mcg | 1,000 mcg synthetic |
| compound | 600 mcg for pregnant women (NIH) | |
| Vitamin B₆ | 2 mg | 100 mg |
| Vitamin B₁₂ | 6 mcg | Not Determined |

Information in this table was derived from the Council for Responsible Nutrition (2001)
*Recommended Daily Intake (RDI) is the value established by the FDA for use in labeling.
**Upper Limit (UL) is the upper limit of intake considered safe for use by adults (unless otherwise specified, the UL combines all potential sources of nutrient).

Another ingredient that can be a preferred non-controlled component of a dosage form according to the invention is vitamin B₆. The term “vitamin B₆” is used in the present description in a manner consistent with its understood meaning in the vitamin and medical arts. The term means compounds generally understood to provide vitamin B₆ efficacy in its administered form or as a derivative of the administered form. The compounds are understood to include pyridoxine compounds such as pyridoxine hydrochloride or any other of the vitamins of the B₆ complex (i.e., codecarboxylase, pyridoxal hydrochloride, or pyridoxamine dihydrochloride) or any precursors or analogues thereof which would give rise to vitamin B₆-like activity. Vitamin B₆ is a known cofactor in metabolizing homocysteine, and therefore can reduce serum homocysteine in the treatment or prevention of cardiovascular disease.

The daily oral dosage of vitamin B₆ administered according to the invention can be any useful and therapeutic amount, and in certain preferred embodiments of the invention can exceed RDI for vitamin B₆. The RDI for vitamin B₆ is 2 mg per day. Exemplary dosage forms according to the inventive methods and formulations can include 2 mg per day or more, e.g., can be at least 5 mg or 10 mg vitamin B₆ per day, or even up to or exceeding 20, 50, or 100 milligrams vitamin B₆ per day.

Still another ingredient that can be a preferred non-controlled release component of a dosage form according to the invention is vitamin B₁₂. The term “vitamin B₁₂” is used in the present description in a manner consistent with its understood meaning in the vitamin and medical arts. The term means compounds generally understood to provide vitamin B₁₂ efficacy in its administered form or as a derivative of the administered form. The compounds are understood to include cobalamin compounds such as hydroxycobalamin, methylcobalamin, and cyanocobalamin or any other substances or any precursors or analogues thereof which would give rise to vitamin B₁₂-like activity. Vitamin B₁₂ is a known cofactor in metabolizing homocysteine, and therefore can reduce serum homocysteine in the treatment or prevention of cardiovascular disease.

The daily oral dosage of vitamin B₁₂ administered according to the invention can be any useful and therapeutic amount, and in certain preferred embodiments of the invention may exceed the recommended daily intake, RDI, for vitamin B₁₂. The RDI for vitamin B₁₂ is 6 mcg per day. Exemplary daily dosages of vitamin B₁₂, according to dosage forms and methods of the invention, can exceed 6 mcg, e.g., may be from about 20 mcg, 50 mcg, 100 mcg, or 150 micrograms vitamin B₁₂ up to about 1000 micrograms B₁₂.

Other ingredients that can be preferred non-controlled release ingredients of a dosage form according to the invention include vitamins D, K, A, and C; calcium; riboflavin (vitamin B₂); boron; and iron; among other vitamins and minerals.

The term “vitamin D” is used in a manner consistent with its understood meaning in the vitamin and medical arts. The term means compounds generally understood to provide vitamin D efficacy in its administered form or a derivative of its administered form. Exemplary vitamin D compounds include vitamin D₁, vitamin D₂, vitamin D₃, or vitamin D₄, or any precursor or analogue to any of these vitamins that would give rise to vitamin D-like activity after administration.

The term “vitamin K” is used in a manner consistent with its understood meaning in the vitamin and medical arts. The term means compounds generally understood to provide vitamin K efficacy in its administered form or a derivative of its administered form. Exemplary vitamin K compounds include vitamin K₁, vitamin K₂, vitamin K₃, vitamin K₄, vitamin K₅, vitamin K₆ and vitamin K₇, or any precursor or analogue to any of these vitamins (such as the naphthaquinones), which would give rise to vitamin K-like activity after administration.

The term “vitamin A” is used in a manner consistent with its understood meaning in the vitamin and medical arts. The term means compounds generally understood to provide vitamin A efficacy in its administered form or a derivative of its administered form. The compounds are understood to include β-ionone derivates possessing qualitatively the biological activity of retinol, retinol, β-carotene, and salts thereof such as the acetate or palmitate salts or any precursors or analogues thereof which would give rise to vitamin A-like activity after administration.

The term “vitamin C” is used in a manner consistent with its understood meaning in the vitamin and medical arts. The term means compounds generally understood to provide vitamin C efficacy in its administered form or a derivative of its administered form. Vitamin C compounds include vitamin C in any of its forms (e.g., salts of ascorbic acid) or any precursor or analogue which would give rise to vitamin C-like activity after administration. A preferred form of vitamin C is ascorbic acid.

Any other vitamin or mineral discussed herein also is used in a manner consistent with their understood meanings in the vitamin and medical arts. The terms identify compounds generally understood to provide vitamin or mineral efficacy in an administered form or as a derivative of an administered form. Examples of these terms include calcium, magnesium, thiamine, riboflavin, boron, iron, and vitamin E. The selected amount of any one or more of these vitamins and minerals may be administered preferably in a single dose.

As used herein, the term “oral dosage form” refers to any suitable product formulation that can be consumed orally to deliver vitamins and minerals as described herein. Examples include tablets (e.g., compressed tablets) and capsules including soft gelatin or hard gelatin capsules. As described, the dosage form includes a controlled-release portion and a non-controlled release portion, with the controlled release portion containing therapeutic magnesium and the non-controlled release portion containing one or more additional vitamins or minerals such as a folic acid compound, vitamin B₆, and vitamin B₁₂.

One specific embodiment of the invention is a dosage form that contains controlled release magnesium in combination with non-controlled release folic acid compound, vitamin B₆, and vitamin B₁₂. One preferred such dosage form can include from 100 to 600 milligrams controlled release elemental magnesium, preferably controlled release magnesium oxide, e.g., from 200 to 800 milligrams controlled release magnesium oxide. Such a preferred dosage form can include folic acid compound, vitamin B₆, and vitamin B₁₂, in any useful or desired amounts, e.g.,: from about 0.5 to about 5 milligrams immediate release folic acid compound, from about 5 to about 50 mg immediate release vitamin B₆, and from about 50 to about 1000 micrograms immediate release vitamin B₁₂.

The controlled release portion of a dosage form according to the invention can be formulated and constructed using any controlled release dosage form construction that results in a desired controlled rate of release of therapeutic magnesium into the digestive system. Many such controlled release dosage form constructions are known and will be understood. An advantage of a controlled release dosage form is that relatively high local concentrations of magnesium compound in the gastrointestinal tract (as would result from immediate release of magnesium) are avoided, due to a controlled, gradual distribution of magnesium compound throughout the gastrointestinal tract, preferably starting in the stomach. Exemplary dosage forms may be capsules or compressed tablets that begin to release therapeutic magnesium in the stomach and gradually release the magnesium compound over a period of hours.

Various different types of controlled release dosage forms may be useful. Typically, a controlled release dosage form can include therapeutic magnesium in combination with one or more additional pharmaceutically acceptable ingredients such as a binder, a controlled release agent, or a coating (e.g., a water-insoluble coating). The binder can be used, if needed, to maintain the shape and form of a dosage form or portion thereof, in general to hold ingredients together. A controlled release agent can be used to control the rate of release of an active ingredient, e.g., by slowing dissolution of a dosage form or a portion thereof. A coating can be used also to control or moderate release of an active ingredient, generally by requiring the coating to be dissolved before active ingredient is released from a coated portion of a dosage form such as a particle. The controlled release dosage form may include any of these components structured in a useful controlled-release form, such as in the form of particles, coated particles, a capsule, a compressed tablet, a compressed layer, combinations of these, etc., as desired.

Examples of useful binders for use in dosage form, e.g., for holding together a particle, a compressed tablet, or a compressed layer of a dosage form, may include calcium sulfate, calcium carbonate, microcrystalline cellulose, hydroxypropyl methylcellulose, starches, lactose, sucrose, mannitol, sorbitol, polyvinylpyrrolidone, methylcellulose, sodium carboxymethylcellulose, ethylcellulose, polyacrylamides, polyvinyloxoazolidone, and polyvinyl alcohols.

Controlled release agents are ingredients known in the pharmaceutical arts, that can be added to a dosage form such as a compressed tablet or particle, to control release of an active ingredient. For example, a controlled release agent present in combination with a binder and therapeutic magnesium can slow the speed of erosion of the binder, thereby causing a slow and gradual erosion of a compressed dosage form and a gradual release of therapeutic magnesium. Examples of useful controlled release agents (or “sustained” release agents) include generally water-soluble polymeric materials such as hydrophilic cellulosic compounds including high viscosity hydroxypropyl methylcellulose (e.g., Methocel K100 MCR from Dow), methyl cellulose, sodium carboxymethylcellulose, etc.; polyvinyl acetate; cellulose acetate; cellulose acetate phthalate; Carrageenan; hydrogenated vegetable oil; waxes (microcrystalline, white, yellow); glyceryl monostearate; acrylic polymers (e.g., Carbopol 934 and 971 from BF Goodrich); polymethacrylates (e.g., Eudragit RL and RS from Degussa); ethylcellulose; and combinations of these or others.

Other ingredients may also be included in various other dosage form constructions, to control release of an active ingredient. In one specific example, a water-insoluble material may be included in a dosage form as a coating of particles that contain (or consist of) therapeutic magnesium. The water-insoluble material may be any material that effectively controls release of the magnesium compound of a coated particle. Many such water-insoluble materials are well known, and include generally hydrophobic materials such as those described above as being useful as controlled or sustained release agents such as cellulose acetate, cellulose acetate phthalate, hydrogenated vegetable oil, waxes, polymethacrylates, and ethyl cellulose.

Particles, capsules, or compressed tablets may also include conventional inactive materials such as bulking agents, fillers, diluents, compression aids, coatings, disintegrants, lubricants, flavorings, and coloring agents.

Particular embodiments of dosage forms of the invention include compressed dosage forms that contain, e.g., a compressed tablet or a compressed layer or portion of a tablet that contains therapeutic magnesium. The compressed tablet, layer, or portion, dissolves gradually over a desired period of time to gradually and slowly release therapeutic magnesium. The release of therapeutic magnesium compound from a compressed dosage form can be gradual, based on erosion (by gastrointestinal fluid) of the mass of a compressed “matrix” that is constructed to include the magnesium and other ingredients that effect a controlled release. The matrix may include, for instance, a binder and a controlled release agent, or particles coated with a water-insoluble coating and compressed to form a compressed tablet, layer, or other portion. Compressed dosage forms may also optionally include other non-active ingredients such as a compression aid (e.g., lactose); a disintegrant such as crospovidone (cross-linked polyvinylpyrrolidone), a modified starch (e.g., sodium carboxymethyl starch), or a modified cellulose gum (e.g., croscarmellose sodium type A.N.F.); and a lubricating agent such as magnesium stearate or stearic acid.

Other embodiments of dosage forms useful according to the invention include capsules that contain therapeutic magnesium in a controlled release form. Capsules can include multiple particles that contain therapeutic magnesium compound for controlled release. The particles can optionally include one or more of a binder or a controlled release agent. The particles may also optionally be coated with a desired coating such as a water-insoluble coating, also to affect release of the therapeutic magnesium. One such capsule dosage form can be based on a plurality of small particles that include a therapeutic magnesium compound such as magnesium oxide, optionally blended with one or more inactive material such as a binder, and preferably coated with a water-insoluble material. Inactive, pharmaceutical acceptable ingredients may also be present in the particles, e.g. materials conventionally used in the preparation of pharmaceutically compositions such as fillers (e.g., lactose), glidants (e.g. silica), or lubricants (e.g. magnesium stearate), but are not required and may be excluded.

As discussed in the present description, dosage forms of the invention can also preferably include non-controlled, preferably immediate, release ingredients in the form of a non-controlled release portion of a dosage form. These can be included in the dosage forms in any construction that allows for non-controlled, e.g., immediate, release of the ingredients (e.g., folic acid compound, vitamin B₆, and vitamin B₁₂). Generally, a non-controlled release portion of a dosage form may contain ingredients such as those used to produce a controlled release portion, such as a binder and other optional pharmaceutically acceptable ingredients or processing aids. Unlike a controlled release portion of a dosage form, a non-controlled release portion does not necessarily require the use of a controlled or sustained release agent or a water-insoluble ingredient (e.g., coating), to effect a controlled release profile. A non-controlled release portion of a dosage form may take the form of a compressed tablet or layer or portion of a compressed tablet, or particles of a capsule, etc.

In a capsule, a non-controlled release portion can be in the form of non-controlled release particles, or in the form of a miniaturized tablet (e.g., compressed tablet). Either can be included in a capsule in to provide a desired dosage of non-controlled release (e.g., immediate release) ingredients such as immediate release vitamins and minerals (e.g., folic acid compound, vitamin B₆, and vitamin B₁₂). Non-controlled release particles can be prepared by any useful method, as will be understood, for example by combining ingredients such as vitamins or minerals with a binder, optional other pharmaceutical ingredients such as a filler, glidant, or lubricant, etc. Such particles can be combined with controlled release particles and encapsulated. A miniaturized tablet can be prepared by any useful method of preparing a tablet (e.g., compressed tablet), and the tablet can be combined with controlled release particles and encapsulated.

One specific example of a compressed tablet dosage form is a bi-layer tablet having one controlled release layer and one non-controlled release layer. The controlled release layer includes a compressed layer that contains therapeutic magnesium compound (preferably magnesium oxide) in a binder (e.g., hydroxypropyl methylcellulose, polyvinyl pyrrolidone, starch, or a combination of these), and also a controlled or sustained release agent such as high viscosity hydroxypropyl methyl cellulose, ethylcellulose, or the like. The ingredients can be combined by wet mixing or by dry mixing, optionally granulated and dried (if wet mixed) and then compressed to form a controlled release portion of a tablet. The non-controlled release layer is a compressed layer containing folic acid compound, vitamin B₆, and vitamin B₁₂, in a bulking agent or filler. Upon ingestion, the bi-layer tablet enters the stomach where the non-controlled release layer disintegrates rapidly and the folic acid, vitamin B₆, and vitamin B₁₂ become dispersed in the stomach and enter the bloodstream. Also upon ingestion, the matrix of the controlled release layer may preferably begin to disintegrate at its outer surface. The surface gradually continues to disintegrate and release therapeutic magnesium in a controlled release fashion, e.g., over a period of hours.

Dosage forms of the invention, including a controlled release portion and preferably a non-controlled release portion, can be prepared by techniques that are available to those of skill in the pharmaceutical arts. Some general steps can include mixing ingredients to prepare a dry or wet mixture, granulation (e.g., wet granulation) or other methods of particle formation, compression to form tablets or portions of tablets, and coating techniques.

According to one general step useful for preparing a controlled release portion of a dosage form according to the invention, therapeutic magnesium and any other ingredients may be combined as a dry powder, with a controlled or sustained release agent. The dry powder mixture can be compressed into a controlled release tablet or a portion of a controlled release tablet. For instance, a dry mixture containing therapeutic magnesium, binder, and controlled release agent, can be combined to a uniform powder and compressed into a compressed form having controlled release properties.

Alternatively, other useful steps may include wet granulation methods. By wet granulation, a therapeutic magnesium compound and other optional ingredients can be wet granulated to form particles that may be used to form a compressed tablet, a layer or other portion of a compressed tablet, a capsule, etc. The particles may or may not contain a controlled or sustained release agent. If a controlled or sustained release agent is not included in the particles, a water-insoluble coating can be coated on the surface of the particles to effect a controlled release, and the particles may be formed into a compressed tablet or a portion of a compressed tablet, or may be filled in a capsule. If a controlled or sustained release agent is included in the particles, the particles may be compressed into a tablet or used in a capsule without being first coated with a water-insoluble coating.

A water-insoluble coating, if used, may by prepared and applied to a dosage form of a portion thereof, such as a particle, by known coating methods. For example, a water-insoluble coating may be provided in the form of a solution or dispersion of water-insoluble coating material in organic or aqueous solvent. Suitable organic solvents include, for example lower alkanols such as ethanol or isopropanol, lower alkyl ketones such as acetone, lower alkyl ethers such as diethyl ether, and mixtures thereof. The solution or dispersion can be applied to particles, e.g., by spray coating, in an amount that will provide desired release properties. The coated particles can be filled into capsules, e.g. a conventional pharmaceutically acceptable hard gelatine capsule, or may be compressed into a tablet or portion of a tablet.

The dosage methods and formulations of the invention used to treat or prevent any one or more of a general magnesium deficiency, a degenerative bone disease such as osteoporosis, diabetes (e.g., to improve glycemic control in a diabetic patient), or cardiovascular disease such as hypertension. Such a method can preferably include administering a dosage form as described herein, that contains a daily dosage of a therapeutic magnesium compound, preferably magnesium oxide, in a described amount; in combination with folic acid compound in an amount that is equal to or greater than the RDI, e.g., greater than 400 mcg per day, preferably greater than 500 mcg per day, e.g., greater than 1000 or 2000 mcg per day; optionally and preferably also in combination with vitamins B₆ and B₁₂. Certain preferred embodiments for use in such methods can include an amount of vitamin B₆, e.g., to provide a daily dosage of an amount in the range from about 5, 10, or 20 mg to about 50 mg per day vitamin B₆; and vitamin B₁₂, e.g., to provide a daily dosage of an amount in the range from about 100 or 150 mcg to about 1000 mcg per day vitamin B₁₂. Other vitamin, mineral, or active pharmaceutical compounds can also be included, especially in the non-controlled release portion of a dosage form. According to certain preferred embodiments of the invention, however, such dosage forms (multivitamin formulations) may exclude and do not include any additional active vitamins, minerals, or pharmaceutical compounds.

The following tables describe potential formulations for tablet dosage forms containing controlled release therapeutic magnesium and non-controlled release folic acid. Different tables relate to different mechanisms for controlled release portions of a tablet. A general concept is for a bi-layer tablet. Each example includes two tables. The first table describes a general concept with exemplary ranges and types of ingredients, and the second table describes a specific example of the general concept including specific amounts and types of ingredients.

In each general example, a controlled (sustained/extended) release layer can contain useful amounts of therapeutic magnesium, e.g., 580 mg of magnesium oxide (350 mg of elemental magnesium). An immediate-release layer can contain useful amounts of other vitamins and minerals, e.g., 10 mg of vitamin B₆, 1 mg of folic acid, and 0.12 mg (120 mcg) of vitamin B₁₂. Other sources of magnesium could be used, but the magnesium oxide has the highest concentration of magnesium and, therefore, allows the lowest amount of material to be used, which will keep the size of the tablet down.

Any of several different excipients can be used for each layer, and the tables will show a variety, but not all, of the options. The described tablets can be coated for cosmetic reasons, e.g., with a thin film to add color and cover the bi-layer construction, even though such a coating is not described.

The tables list the materials by layer. The top portion of each table describes magnesium (oxide) and excipients in a controlled-release layer. The lower portion of each table describes vitamins and excipients in a non-controlled release layer. The percentages listed will be per layer, so the values will total 200%.

### EXAMPLES 1 AND 2

### Hydrophilic Matrix Tablets Using Hydroxypropyl Methylcellulose (HPMC)

| | PERCENT |
|---|---|
| INGREDIENTS | OF DOSE |
| Magnesium Oxide (580 mg/tablet) | TBD |
| Sustained Release Agent (Hydroxypropyl | 5-35 |
| Methylcellulose, High Viscosity) | |
| Binder (Hydroxypropyl Methylcellulose, | 0.5-10 |
| Polyvinylpyrrolidone, Starch, etc.) | |
| Antiadherent (Silicon Dioxide, Starch, Talc, etc.) | 0.1-10 |
| Glidant/Flow Aid (Magnesium Stearate, Silicon Dioxide, | 0.1-5 |
| Talc, etc.) | |
| Lubricant (Hydrogenated Vegetable Oil, Magnesium | 0.1-4 |
| Stearate, Stearic Acid, etc.) | |
| Vitamin B₆ (10 mg) Folic Acid (1 mg) Vitamin | TBD |
| B₁₂ (120 mcg) | |
| Bulking Agent (Filler/Diluent) (Dicalcium Phosphate, | 0-90 |
| Lactose, Microcrystalline Cellulose, Starch, etc.) | |
| Antiadherent (Silicon Dioxide, Starch, Talc, etc.) | 0.1-10 |
| Glidant/Flow Aid (Magnesium Stearate, Silicon | 0.1-5 |
| Dioxide, Talc, etc.) | |
| Lubricant (Hydrogenated Vegetable Oil, Magnesium | 0.1-4 |
| Stearate, Stearic Acid, etc.) | |
| Magnesium Oxide (580 mg/tablet) | 83.0 |
| Sustained Release Agent: Hydroxypropyl | 8.0 |
| Methylcellulose, High Viscosity | |
| Binder: Hydroxypropyl Methylcellulose | 5.0 |
| Antiadherent & Glidant: Talc | 3.0 |
| Lubricant: Magnesium Stearate | 1.0 |
| Vitamin B₆ (10 mg) Folic Acid (1 mg) Vitamin | 8.5 |
| B₁₂ (120 mcg) | |
| Bulking Agent: Lactose | 64.7 |
| Bulking Agent: Starch | 24.3 |
| Antiadherent & Glidant: Silicon Dioxide | 2.0 |
| Lubricant: Magnesium Stearate | 0.5 |

### EXAMPLES 3 AND 4

### Tablets with Water-Insoluble, Ethyl Cellulose-Coated Particles

| | PERCENT |
|---|---|
| INGREDIENTS | OF DOSE |
| Magnesium Oxide (580 mg/tablet) | TBD |
| Sustained Release Agent (Ethylcellulose) | 5-25 |
| Bulking Agent (Filler/Diluent) (Dicalcium Phosphate, | 0-90 |
| Lactose, Microcrystalline Cellulose, Starch, etc.) | |
| Disintegrant (Croscarmellose Sodium, Sodium Starch | 0.3-8 |
| Glycolate, etc.) | |
| Antiadherent (Silicon Dioxide, Starch, Talc, etc.) | 0-10 |
| Glidant/Flow Aid (Magnesium Stearate, Silicon Dioxide, | 0-5 |
| Talc, etc.) | |
| Lubricant (Hydrogenated Vegetable Oil, Magnesium | 0-4 |
| Stearate, Stearic Acid, etc.) | |
| Vitamin B₆ (10 mg) Folic Acid (1 mg) Vitamin B₁₂ | TBD |
| (120 mcg) | |
| Bulking Agent (Filler/Diluent) (Dicalcium Phosphate, | 0-90 |
| Lactose, Microcrystalline Cellulose, Starch, etc.) | |
| Antiadherent (Silicon Dioxide, Starch, Talc, etc.) | 0.1-10 |
| Glidant/Flow Aid (Magnesium Stearate, Silicon Dioxide, | 0.1-5 |
| Talc, etc.) | |
| Lubricant (Hydrogenated Vegetable Oil, Magnesium | 0.1-4 |
| Stearate, Stearic Acid, etc.) | |
| Magnesium Oxide (580 mg/tablet) | 74.3 |
| Sustained Release Agent: Ethylcellulose | 14.0 |
| Bulking Agent: Microcrystalline Cellulose | 10.0 |
| Disintegrant: Croscarmellose Sodium | 1.0 |
| Antiadherent: Silicon Dioxide | 0.5 |
| Lubricant: Magnesium Stearate | 0.2 |
| Vitamin B₆ (10 mg) Folic Acid (1 mg) Vitamin B₁₂ | 4.5 |
| (120 mcg) | |
| Bulking Agent: Microcrystalline Cellulose | 94.5 |
| Lubricant: Magnesium Stearate | 1.0 |

### EXAMPLES 5 AND 6

### Tablets with Water-Insoluble Acrylic Polymers

| | PERCENT |
|---|---|
| INGREDIENTS | OF DOSE |
| Magnesium Oxide (580 mg/tablet) | TBD |
| Sustained Release Agent (Acrylic Polymers) | 5-50 |
| Bulking Agent (Filler/Diluent) (Dicalcium Phosphate, | 0-90 |
| Lactose, Microcrystalline Cellulose, Starch, etc.) | |
| Binder (Hydroxypropyl Methylcellulose, | 0.5-10 |
| Polyvinylpyrrolidone, Starch, etc.) | |
| Plasticizer (Triethyl Citrate, Dibutyl Sebacate, etc.) | 0-10 |
| Antiadherent (Silicon Dioxide, Starch, Talc, etc.) | 0-10 |
| Glidant/Flow Aid (Magnesium Stearate, Silicon Dioxide, | 0-5 |
| Talc, etc.) | |
| Lubricant (Hydrogenated Vegetable Oil, Magnesium | 0-4 |
| Stearate, Stearic Acid, etc.) | |
| Vitamin B₆ (10 mg) Folic Acid (1 mg) Vitamin B₁₂ | TBD |
| (120 mcg) | |
| Bulking Agent (Filler/Diluent) (Dicalcium Phosphate, | 0-90 |
| Lactose, Microcrystalline Cellulose, Starch, etc.) | |
| Antiadherent (Silicon Dioxide, Starch, Talc, etc.) | 0.1-10 |
| Glidant/Flow Aid (Magnesium Stearate, Silicon Dioxide, | 0.1-5 |
| Talc, etc.) | |
| Lubricant (Hydrogenated Vegetable Oil, Magnesium | 0.1-4 |
| Stearate, Stearic Acid, etc.) | |
| Magnesium Oxide (580 mg/tablet) | 65.2 |
| Sustained Release Agent: Ammoniomethacrylate | 18.0 |
| copolymers | |
| Bulking Agent: Dicalcium Phosphate | 15.0 |
| Antiadherent & Glidant: Talc | 1.0 |
| Lubricant: Stearic Acid | 0.8 |
| Vitamin B₆ (10 mg) Folic Acid (1 mg) Vitamin B₁₂ | 3.5 |
| (120 mcg) | |
| Bulking Agent: Microcrystalline Cellulose | 50.0 |
| Bulking Agent: Dicalcium Phosphate | 43.5 |
| Antiadherent & Glidant: Silicon Dioxide | 2.0 |
| Lubricant: Stearic Acid | 1.0 |

1. A method of administering magnesium, the method comprising orally administering a dosage form comprising
non-controlled release folic acid compound, vitamin B₆, and vitamin B₁₂, and
controlled release magnesium.

2. The method of claim 1 wherein the dosage form comprises controlled release magnesium oxide.

3. The method of claim 2 wherein the dosage form comprises from 200 to 800 milligrams controlled release magnesium oxide.

4. The method of claim 1 wherein the dosage form comprising
greater than 500 micrograms folic acid compound, and
from 100 to 600 milligrams controlled release elemental magnesium.

5. The method of claim 1 wherein the dosage form comprises
from 500 to 1500 micrograms folic acid compound, and
from 300 to 600 milligrams controlled release elemental magnesium.

6. The method of claim 1 comprising administering a single dosage form per day.

7. The method of claim 1 wherein the dosage form contains active ingredients consists essentially of:
controlled-release magnesium oxide,
immediate-release folic acid,
immediate-release vitamin B₁₂, and
immediate release vitamin B₆.

8. The method of claim 7 comprising administering a single dosage form per day.

9. The method of claim 1 wherein the dosage form contains active ingredients consisting essentially of:
from about 100 to about 600 milligrams controlled-release elemental magnesium oxide,
from about 0.5 to about 5 milligrams immediate release folic acid compound,
from about 5 to about 50 mg immediate release vitamin B₆, and
from about 50 to about 1000 micrograms immediate release vitamin B₁₂.

10. The method of claim 1 wherein at least about 80 percent of the magnesium is released over a period of time in the range from 6 to 24 hours.

11. The method of claim 1 wherein at least about 80 percent of the magnesium is released over a period of time in the range from 8 to 12 hours.

12. The method of claim 1 wherein
the dosage form comprises non-controlled release folic acid, and
at least 80 percent of the non-controlled release folic acid is released within 60 minutes.

13. The method of claim 1 wherein
the dosage form comprises non-controlled release folic acid, vitamin B₁₂, and vitamin B₆, and
at least 80 percent of the non-controlled release folic acid, vitamin B₁₂, and vitamin B₆, is released within 60 minutes.

14. The method of claim 1 wherein the oral dosage form is a bi-layer tablet comprising
a non-controlled release layer comprising folic acid compound, vitamin B₆, and vitamin B₁₂, and
a controlled release layer comprising therapeutic magnesium.

15. The method of claim 14 wherein the controlled release layer is a compressed layer comprising magnesium oxide, binder, and sustained release agent.

16. The method of claim 1 comprising administering the formulation to treat or prevent magnesium deficiency.

17. The method of claim 1 comprising administering the formulation to treat or prevent a condition selected from the group consisting of a degenerative bone disease, cardiovascular disease, and diabetes.

18. The method of claim 1 comprising administering the formulation to treat or prevent osteoporosis.

19. An oral dosage form comprising
non-controlled release folic acid compound, vitamin B₆, and vitamin B₁₂, and
controlled release magnesium.

20. The dosage form of claim 19 wherein the therapeutic magnesium is magnesium oxide.

21. The dosage form of claim 19 wherein the dosage form comprises from 200 to 800 milligrams controlled release magnesium oxide.

22. The dosage form of claim 19 comprising greater than 400 micrograms folic acid compound.

23. The dosage form of claim 19 wherein the dosage form is a bi-layer tablet comprising
a non-controlled release layer comprising folic acid compound, vitamin B₆, and vitamin B₁₂, and
a controlled release layer comprising magnesium oxide.

24. The dosage form of claim 23 wherein
the non-controlled release layer comprises
from 0.5 to 5 milligrams folic acid compound,
from 5 to 50 milligrams vitamin B₆, and
from 50 to 1000 micrograms vitamin B₆, and
the controlled release layer comprises from 200 to 800 milligrams magnesium oxide.

25. The dosage form of claim 19 wherein the controlled release layer is a compressed layer comprising magnesium oxide, binder, and sustained release agent.

26. The dosage form of claim 19 wherein the dosage form contains active ingredients consists essentially of:
controlled-release magnesium oxide,
immediate-release folic acid,
immediate-release vitamin B₁₂, and
immediate release vitamin B₆.

27. The dosage form of claim 19 wherein the dosage form contains active ingredients consisting essentially of:
from about 200 to about 800 milligrams controlled-release magnesium oxide,
from about 0.5 to about 5 milligrams immediate release folic acid compound,
from about 5 to about 50 mg immediate release vitamin B₆, and
from about 50 to about 1000 micrograms immediate release vitamin B₁₂.
